(19) United States
(12) Patent Application Publication
(10) Pub. No.: US 2014/0276727 A1
(43) Pub. Date: Sep. 18, 2014

(54) Reliable Low Profile Junction for Electrode Array

(71) Applicant: MEDTRONIC ARDIAN LUXEMBOURG S.A.R.L., Luxembourg (LU)

(72) Inventor: Carlo Guala, Roncadelle (IT)

(73) Assignee: MEDTRONIC ARDIAN LUXEMBOURG S.A.R.L., Luxembourg (LU)

(21) Appl. No.: 13/803,063

(22) Filed: Mar. 14, 2013

(51) Int. Cl. A61B 18/14; A61B 18/18

(52) U.S. Cl. CPC A61B 18/1492; A61B 18/18
USPC 606/33; 606/41

(57) ABSTRACT

A device and method for retrieving an electrode array. The medical device includes a catheter defining a proximal end, a distal end, and a lumen therethrough. A control wire slidably receivable within the lumen is included, the control wire defining a proximal end and a distal end, the distal end of the control wire defining an enlarged portion. A treatment portion receivable within the lumen is included, the treatment portion including a proximal portion adapted to receive the enlarged portion at the distal end of the control wire.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method and system for deploying and retrieving a treatment portion of an energy delivery catheter.

### BACKGROUND OF THE INVENTION

With the increasing versatility of modern medical devices to traverse the smallest passages within the body, and treat the smallest tissues and blood vessels, comes an increasing risk of failure of these medical devices owing to their small size or weak attachment points. For example, endovascular catheters may include a radially expandable treatment portion affixed to a control wire for expelling the treatment portion from the open distal end of the catheter and for retracting and thereby collapsing the treatment portion into the catheter after a treatment modality is performed. However, because the treatment portion and control wire are often made of metals such as TiNi alloy (nitinol) to capitalize on their pseudo-elastic properties, common joining techniques such as soldering or welding are more difficult to perform successfully than would be the case with other metals such as stainless steel, resulting in a weak point in the junction between the treatment portion and the control wire.

FIG. 1 shows an example of a treatment portion of a neuromodulation catheter system operable to ablate or otherwise inhibit renal nerves. In this example, the distal end of control wire is laser welded to the proximal end of the treatment portion. This welding junction may be susceptible to failure after multiple uses of the assembly, or alternatively, may be more likely to fail owing to operator error when deploying and expanding the treatment portion. Moreover, the welding process may cause imperfections in the control wire, the junction, and/or the proximal end of the treatment portion. Accordingly, what is needed is a reliable and low-profile junction between a treatment portion and a control wire and a method of use thereof.

### SUMMARY OF THE INVENTION

The present invention provides a medical device and method for retrieving an electrode array from within a human patient. The device includes a catheter defining a proximal end, a distal end, and a lumen therethrough. A control wire slidably receivable within the lumen is included, the control wire defining a proximal end and a distal end, the distal end of the control wire defining an enlarged portion. A treatment portion is receivable within the catheter lumen and includes a proximal portion engageable with the enlarged portion at the distal end of the control wire.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of a prior art medical device;

FIG. 2 is a perspective view of an exemplary medical device having a retrievable treatment portion constructed in accordance with the principles of the present invention;

FIG. 3 is a partial cross-sectional disassembled view of the proximal portion of the treatment portion shown in FIG. 2 and the distal portion of the elongate body;

FIG. 4 is a cross-sectional view of the treatment portion shown in FIG. 2 and the distal portion of the elongate body;

FIG. 5 is a side perspective view of a deployed treatment portion shown in FIG. 2 with the sheath pulled back; and

FIG. 6 is a side perspective view showing the treatment portion of FIG. 5 partially retrieved within the sheath.

### DETAILED DESCRIPTION OF THE INVENTION

The present invention provides for a medical device and method of use thereof to advance and retrieve a treatment portion of a catheter. Referring now to the drawings in which like reference designators refer to like elements, there is shown in FIG. 2 an exemplary embodiment of a medical system 10 for diagnosing and treating tissue such as renal, neural, or vascular tissue. Of note, the device components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the system and devices disclosed herein may be included in a variety of different combinations or configurations without departing from the scope and spirit of the invention.

Referring now to FIG. 2, the system 10 generally includes a medical device 12 that may be coupled to a control unit or operating console 14, which may be for example, a multi-channel radiofrequency (RF) signal generator having a display to monitor the progress of a particular procedure or the status of the components of the medical device 12. The medical device 12 may include an elongate body 16 passable through a patient's vasculature and/or proximate to a tissue region for diagnosis or treatment, such as a catheter, sheath, or intravascular introducer, or a combination thereof. The elongate body 16 may define a proximal portion 18 and a distal portion 20, and may further include one or more lumens disposed within the elongate body 16 thereby providing mechanical, electrical, and/or fluid communication between the proximal and distal portions 18, 20 of the elongate body 16, as discussed in more detail below. The elongate body 16 may include reinforcement elements or otherwise be constructed to provide desired degrees of stiffness, flexibility, and/or torque transmission along the length of the body and at discrete locations along the length thereof. For example, the elongate body 16 may include wires, braiding, increased wall-thickness, additional wall layering, sleeves, or other components reinforcing or otherwise supplementing an outer wall or thickness along its length. Discrete portions that may experience significant loading or torque during a particular procedure may also include such reinforcement.

The medical device 12 may further include a diagnostic or treatment portion 22 sized to be receivable and moveable within the lumen of the elongate body 16 and extendable from the distal portion 20. The treatment portion 22 may assess or measure a property or characteristic of a tissue site (e.g., neural activity, tissue composition assessments, tissue contact assessment, or the like) and/or it may deliver or otherwise transmit a therapeutic or diagnostic signal or energy to a tissue site (e.g., electrical energy delivery, tissue ablation, neuromodulation, or the like). The treatment portion 22 may deliver, for example, radiofrequency energy, cryogenic therapy, or the like to a tissue area in proximity to the distal portion 20 of the medical device 12. The treatment portion 22 may further include an expandable member (not shown), for example, a balloon, or other treatment mediums for transferring thermal energy such as cryogenic energy.

The diagnostic or treatment portion 22 may include one or more electrodes 24 or electrically conductive portions of electrodes 24. The electrodes 24 may include variations in their number, arrangement, configuration, or shape and may be constructed from conductive materials such as silver, platinum or gold, for example. The electrodes 24 may be coupled to, welded, or otherwise disposed on one or more electrode arrays 26 disposed near, on, or substantially on the distal end of the elongate body 16 to detect electrical signals between any pair of electrodes (bi-pole) for mapping of electrical activity, and/or for performing other functions such as ablating neural tissue. Moreover, the electrodes 24 may deliver ablation energy across an electrode pair or from independent electrodes 24 when delivering monopolar energy. The electrode arrays 26 may be composed of any flexible material, for example nitinol, may be conductive or non-conductive and may be linear, curvilinear, or any shape such that each array 26 may be expandable and collapsible within the catheter lumen.

The electrode arrays 26 may further define an electrode array assembly or a carrier assembly 28 in which a plurality of electrode arrays 26 are welded or otherwise joined to each other to define a three-dimensional, expandable, and retractable treatment portion 22. For example, as shown in FIG. 2, three electrode arrays 26, each having one electrode 24, are combined together to define the electrode array assembly 28. Each electrode array 26 may be resilient to bias the electrode array assembly 28 in an expanded configuration and facilitate the radial compression or otherwise retraction of each electrode array 26 into an unexpanded configuration. Although three electrode arrays 26 each having one electrode 24 are shown, it is contemplated that any number of electrodes 24, electrode arrays 26, in any shape or size, may define the expandable array assembly of electrodes 28. The treatment portion 22 may also comprise the one shown in FIG. 1, which is described in U.S. patent application Ser. No. 13/613,091, filed Sep. 13, 2012, the disclosure of which is incorporated by reference herein.

The carrier assembly 28 may further define a proximal portion 30 at which the electrode arrays 26 join to define, in part, a basket-like junction proximate the distal end of the catheter lumen. For example, the proximal portion 30 may define a substantially cylindrical structure 32 that extends a predetermined distance into the distal portion 20 of the elongate body 16. In an exemplary configuration, the cylindrical structure 32 is slidable within the lumen and may further define a beveled edge, or as described in more detail below, a plurality of cantilevered fingers 33 (FIG. 3) that are circumferentially disposed about the proximal end of the cylindrical structure 32 that flex inward and are biased in a configuration in which the fingers are pressed against the interior wall of the catheter lumen.

Continuing to refer to FIG. 2, each of the electrodes 24 may be electrically coupled to the RF signal generator 14, which may also be attached to a return or reference electrode 34, such as a conductive pad attached to the back of the patient, to enable the delivery of monopolar ablation energy when desired. While monopolar and bipolar RF ablation energy may be the selected forms of energy to pass through the electrodes 24 of the medical device 12, other forms of ablation energy may be additionally or alternatively emitted from the treatment assembly, including electrical energy, magnetic energy, microwave energy, thermal energy (including heat and cryogenic energy) and combinations thereof. Moreover, other forms of energy that may be applied can include acoustic energy, sound energy, chemical energy, photonic energy, mechanical energy, physical energy, radiation energy and a combination thereof.

The handle assembly 36 of the medical device may include one or more mechanisms or components to facilitate manipulation of the elongate body 16 and the distal treatment portion 22. For example, as shown in FIG. 2, the handle assembly 36 may include a linear actuator 38 providing for the proximal-distal extension and retraction of the elongate body 16. The linear actuator 38 may be movably coupled to a portion of the handle assembly 36 to allow it to slide or otherwise translate in a proximal-to-distal direction, and vice versa. A rotational actuator 40 may also be disposed on or about the handle assembly 36 to facilitate rotation of the elongate body 16 (and thus the distal treatment portion 22, including the expandable array assembly of electrodes 28, discussed above) about a longitudinal axis of the elongate body in two directions.

A control wire 42 may also be provided within the medical device 12, and in a particular configuration, coupled to the handle assembly 36. For example, to facilitate retraction and control of the treatment portion 22, a control wire 42 may be slidably receivable within the lumen of the elongate body 16 and extendable out through the distal end of the lumen at the distal end of the elongate body 16. The control wire 42 may extend proximally to a control knob 44 and distally along the length of the lumen. Rotation of the knob 44 may pull the control wire 42 that, in turn, retrieves the expandable array assembly of electrodes 28 as discussed in more detail below.

In the embodiment shown in FIGS. 2 and 3, the proximal portion 30 may extend a distance away from the distal end of the catheter lumen when the elongate body 16 is retracted proximally from the treatment portion 22. In a particular embodiment, the entire proximal portion 30 may extend distally from the lumen when deployed, or alternatively, may extend partially from the distal end of the lumen such that a portion of the treatment portion 22 remains within the catheter lumen. Electrical conduction is maintained between the treatment portion 22 and the control unit 14 via the control wire 42, including where the engagement element 46 is in contact with the proximal portion 30. Additionally, a plurality of wires (not shown) may be disposed within the lumen and are connected to one or more of the plurality of electrodes 24 to enable activation of the electrode arrays 26.

FIG. 3 illustrates several components of the treatment portion 22 prior to assembly. A plurality of fingers 33 extend proximally from the proximal portion 30 of the treatment portion 22. The fingers are circumferentially arranged and are formed at the same diameter as the proximal portion 30. For example, the treatment portion 22 may be cut from a cylindrical tube to create one or more electrode arrays 26 and fingers 33, while leaving the proximal portion 30 uncut. The outer diameter of the proximal portion 30 and fingers 33 is sized to slidably fit within the catheter lumen. The engagement element 46, which is described in further detail below, will slip through the inner diameter of the proximal portion 30 and fingers 33 at the illustrated stage of assembly. To form a receptacle or socket to receive the engagement element 46, a tubular element 48 is forced over fingers 33, as illustrated by arrows in FIG. 3 and as shown in finished form in FIG. 4. The tubular element 48 may be friction fit, coupled, compression fit, or otherwise engaged to the fingers 33 and is sized to slidably fit within the catheter lumen of elongate body 16. The tubular element 48 may be composed of nitinol, stainless steel, or other like components, and in an example, may further be composed of the same tubing as the treatment portion 22. In an exemplary configuration, the tubular element 48 is at least substantially cylindrical in shape and defines an outer diameter that is substantially the same as the outer diameter of the cylindrical portion of the proximal portion 30. The fingers 33 having been forced radially inward, the luminal space extending through the fingers is reduced in transverse size to slidably receive control wire 42, but not to receive the engagement element 46. A low profile joint between control wire 42 and the treatment portion 22 is thus formed at the proximal end of the proximal portion 30 including a receptacle for the engagement element 46. By avoiding high-temperature joining methods such as welding, brazing or soldering, reliability of the joint is expected to be improved.

Referring now to FIGS. 2 and 3, the control wire 42 may be composed of a super elastic alloy such as nitinol, or any resilient and electrically conductive material, for example, stainless steel. The control wire 42 has an engagement element 46 larger than the diameter of the control wire that may be welded, molded, or otherwise affixed to the distal end of the control wire 42 and is sized and shaped to be receivable in the receptacle formed within the proximal portion 30 of the treatment element 22. Engagement element 46 may comprise an adhesive. For example, an acrylic glue, resin, or gel may be applied to the surface of the control wire 42.

In particular, the electrode arrays 26 may join at the proximal portion 30 and taper inward to define a basket-like junction where the electrode arrays 26 are joined at the proximal portion 30. The engagement element 46 fits through the junction to seat in the receptacle within the proximal portion 30 such that a proximally directed pulling force on the engagement element 46 draws the treatment portion 22 within the lumen of elongate body 16. For example, when the engagement element 46 pulls on the receptacle formed by fingers 33, the electrode arrays 26 are resiliently collapsed from an expanded configuration to an unexpanded configuration constrained within the proximal portion 30.

Referring now to FIG. 4, a coil 50 may be slidably disposed within the catheter lumen of elongate body 16, and extending from a proximal end operably attached within handle assembly 36 to a distal end abutting the tubular element 48. The control wire 42 is slidably received within coil 50. The coil 50 may be composed of a shape memory material, such as nitinol, or any resilient material. The coil 50 may increase the kink resistance of the elongate body 16 and may further increase the flexibility/torqueability of the elongate body. The coil 50 may further define an outer diameter substantially equal to the outer diameter of the tubular element 48, for example, both the tubular element 48 and the coil 50 may define an outer diameter of 0.8 mm. The windings of coil 50 are preferably stacked, meaning that there is no space between adjacent windings.

Referring now to FIG. 5, in this configuration, the elongate body 16, which is a catheter or sheath, has been retracted proximally to illustrate the various components of the medical device 12 and a corresponding method of use. In particular, the method of using the medical device 12 may include advancing the elongate body 16 toward a target tissue region to be treated such as the renal artery or other blood vessel. The treatment portion 22 may be released from the distal end of sheath 16 by applying a proximal force on the sheath 16 from the linear actuator 38 of the handle 36. While sheath 16 is being withdrawn, the treatment portion 22 remains stationary with respect to the surrounding vessel or tissue by the application of distal force to coil 50, which in turn transmits the force to tubular element 48 that is part of the treatment portion 22. As the sheath 16 is retracted, radial constraint is removed from the treatment portion 22, which resiliently transforms into an expanded configuration for treating the target tissue. The operator may then activate the treatment portion 22 to treat the target tissue region. For example, bipolar or monopolar RF ablation energy may be transmitted to a target tissue or tissue region such as the renal nerves.

After the desired treatment is completed, the control wire 42 may be pulled proximally such that the engagement element 46 pulls on the receptacle formed by fingers 33 to draw the treatment portion 22 within the catheter lumen as shown in progress in FIG. 6. Elongate body 16 may be held stationary with respect to the patient while treatment portion 22 is withdrawn.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical device, comprising:
a catheter having a lumen extending between a proximal end and an open distal end;
a control wire slidably receivable within the lumen, the control wire having a proximal end and an enlarged engagement element disposed at a distal end;
a treatment portion collapsibly receivable within the lumen and having, at a proximal portion, a receptacle adapted to receive the enlarged engagement element.

2. The medical device of claim 1, wherein the treatment portion includes an expandable array assembly of electrodes.

3. The medical device of claim 1, wherein the engagement element includes an adhesive.

4. The medical device of claim 1, further including a coil slidably extending through the lumen and having a distal end abutting the proximal portion of the treatment portion.

5. The medical device of claim 4, further including a tubular element coupled to the proximal end of the treatment portion.

6. A method of retrieving a medical device from within a human patient, the method comprising applying proximally-directed pulling force on a control wire extending through a catheter lumen such that an enlarged engagement element disposed at a distal end of the control wire transmits the proximally-directed pulling force to a distally-facing receptacle located in a proximal portion of a treatment portion to thereby draw the treatment portion proximally into the catheter lumen.

7. The method of claim 6, wherein the engagement element is composed of stainless steel.

8. The method of claim 6, wherein the treatment portion includes an expandable array assembly of electrodes.

9. The method of claim 6, wherein the treatment portion includes a plurality of electrode arrays, each array having a common proximal end and a common distal end.

10. The method of claim 9, further including, prior to applying proximally-directed pulling force, transmitting at least one of unipolar radiofrequency energy and bipolar radiofrequency energy from at least one of the electrodes.

11. The method of claim 10, further including ablating a target tissue region.

12. A medical device, comprising:
a catheter defining a proximal end, a distal end, and a lumen therethrough;
a control wire slidably receivable within the lumen, the control wire defining a proximal end and a distal end;
an engagement element mounted on the distal end of the control wire, the engagement element defining an outer diameter larger than a diameter of the control wire;
an expandable array assembly of electrodes receivable within the lumen, the assembly having a proximal portion and a tubular element compression fit thereto to define a distally-facing receptacle sized to receive the engagement element; and
a coil slidably extending through the lumen and having a distal end abutting the tubular element.
